(19) United States
(12) Patent Application Publication
(10) Pub. No.: US 2020/0008657 A1
(43) Pub. Date: Jan. 9, 2020

(54) ENDOSCOPIC DEVICE

(71) Applicants: Alessandro BUDANO, Ciampino (RM) (IT); Cesare HASSAN, Rome (IT)

(72) Inventors: Alessandro BUDANO, Ciampino (RM) (IT); Cesare HASSAN, Rome (IT)

(21) Appl. No.: 16/468,015

(22) PCT Filed: Dec. 11, 2017
(86) PCT No.: PCT/IB2017/057793

§ 371 Date: Jun. 10, 2019

(30) Foreign Application Priority Data
Dec. 12, 2016 (IT) 102016000124851

(51) Int. Cl. A61B 1/00; A61B 1/31

(52) U.S. Cl. CPC A61B 1/00137; A61B 1/31

(57) ABSTRACT

An endoscopy cap is described, said cap having the particular feature of allowing distension of the mucosal surfaces undergoing endoscopic examination without disturbing the lateral vision at all, resulting in a significant reduction in the time needed to carry out the procedure and without negatively affecting the possibility of diagnosis of the various mucosal lesions which may be present. The device may be applied to any endoscope and is made of materials which are biocompatible with the environment in which they will be used.

The present invention relates, in general, to the technical sector of movable devices for endoscopes aimed at improving the accuracy during the examination of neoplastic lesions of the colon.

### BACKGROUND ART

Conventional endoscopy, especially conventional colonoscopy (CC), is regarded as being a standard procedure for identifying neoplasms or various alterations of a pathological and non-pathological nature affecting the hollow organs of the body. Owing to the form of the hollow organs and the difficulties in viewing them, even nowadays it very often happens that a significant number of lesions or potentially pathological forms, such as adenomas and polyps, are overlooked. Anatomically speaking in fact the hollow organs are characterized by the presence of folds, which form physiological restrictions inside which viewing of the alterations and/or lesions is extremely difficult, and in particular this occurs in the case of lesions of the colon, especially adenomas and polyps located in certain parts of the colon, for example the right-hand or proximal colon.

Endoscopies are carried out using an endoscope consisting of a flexible probe with a thickness of a few millimeters and containing a camera at the end which allows viewing of the inside of hollow organs of the body (such as the colon, esophagus, stomach, trachea and bladder). Endoscopes are mainly used in a medical environment in order to identify alterations of the internal surfaces of said hollow organs of the body. Endoscopes are also provided with a light source and a service channel of variable dimensions, useful for performing the operations which are involved in the endoscopic procedure.

The irregular nature of the surface of the colon, which is characterized by the presence of folds, makes it difficult to obtain a perfect view of the surface and its structural characteristics, especially in the vicinity of said folds. In order to be able to conduct this examination more efficiently, often technical measures which allow provisional and temporary distension of the surface of the colon have been adopted, these measures consisting for example in the use of caps with flexible flanges to be positioned on the end of the endoscope or the like. The flanges, once opened along the axis of the endoscope as a result of the backward pulling force exerted by the endoscopy operator, cause distension of the surface with which the flanges make contact and allow a planar vision of the surface concerned, with a potential advantage as regards the identification of lesions (i.e. detection rate). The use of a simple cap made of biocompatible plastic is also very widespread, said cap being positioned in this case too on the free end of the endoscope. The use of these caps of transparent plastic hitherto has improved the viewing of the front surface by the camera inside the hollow organs and in particular the colon, ensuring for example that a standard distance is maintained from the lens and allowing optimization of the focal length. The colonoscopy performed with the aid of these caps is called cap-assisted colonoscopy (CAC).

The caps are therefore small hollow cylinders of biocompatible material, which is preferably plastic and transparent, to be positioned on the end of the endoscope in order to obtain distension of the physiological restrictions (folds) which are present on the surface of the hollow organs, such as to improve the visibility in areas which otherwise would not be accessible during the procedure.

The caps used hitherto may be grouped into two general categories, depending on the function which they are intended to perform and their actual positioning: a first group which is mounted on the end of the endoscope in front of the camera (called “cap”) and a second group (called “endocuff” or “endoring”) which is again mounted on the free end of the endoscope, but behind the camera.

The caps mounted in front of the camera are again simple cylinders of preferably plastic material able to distend the folds or mucosa areas situated more directly facing the lens of the said camera (FIG. 1).

The caps mounted behind the camera again have a cylindrical form and are made of preferably plastic material in the same way as the caps mentioned above, but are also provided with additional movable surfaces of varying shape, number and size, called “arms” or “flanges” (i.e. endocuffs) (FIG. 2).

It is known, however, that the caps which are currently used in endoscopy have a several major drawbacks. For example, those which are mounted in front of the camera, owing to the presence of material which infiltrates between the surface to be examined and the lens of the camera, result in a substantial reduction in the amplitude of the visual field of the endoscope used for the procedure and a consequent limited visibility of the area not immediately facing the axis of the camera, where there is no infiltration of material deforming the image with its intrinsic reflective/refractive power. This effect results in a significant reduction in the visual field to only the front area of the endoscope and, consequently, a significant increase in the examination time and the need for continuous displacement of the endoscope tip in order to inspect the entire accessible surface.

The caps which are instead mounted behind the camera, owing to the presence of the additional surfaces or flanges which rub against the mucosal surface to be examined in order to cause distension of the mucosal folds, may cause as a result of friction more or less evident lesions on the mucosa of the hollow organ being examined. If, in order to avoid this potential collateral effect, the preferably plastic or silicone biocompatible material which forms the additional surfaces is made with less rigidity to avoid causing lesions, it may very well be that it does not manage to ensure a suitable degree of distension of the folds. Moreover, inside the right-hand colon, since the lumen of the viscera is bigger in size than the endoring or endocuff, the endoscopy operator is obliged to distend the four walls one at a time, thus prolonging the examination time.

A further drawback of the caps used nowadays in endoscopy is the possible accumulation, inside the volume surrounded by the cap and the tip of the endoscope, of fecal material which reduces the visual capacity during examination. Moreover, the lateral surface of the cap, i.e. the portion of plastic material which acts as a spacer between the circular opening of the cap (situated on the end of the tip) and the surface of the camera lens (positioned at a given distance from the side surface of the cylinder forming the said cap), represents interfering material which deforms the image and prevents perfect viewing (lateral visibility) of the anatomical and functional details of the part of the mucosa being examined by the said endoscopy operator. In fact, all the caps which are hitherto available result in a substantial reduction of the quality and definition of the image on the lateral part of the visual field, where it is known nowadays that a high quality and definition of the endoscopic image is essential in order to identify neoplastic lesions of the colon.

As a result, therefore, the caps which are currently used in endoscopy do not solve in an effective and definitive manner the problem of viewing alterations or lesions positioned in areas which are not superficially planar and homogeneous and are difficult to access inside the hollow organs.

There therefore exists the need to provide a cap device able to improve viewing in endoscopy procedures, especially during distension of the folds of the hollow organs, in a simple and reliable manner without adversely affecting the clarity and definition of the endoscopic image.

### DESCRIPTION OF THE FIGURES

FIG. 1 shows a photograph of a cap of the prior art, which is positioned in front of the camera of the endoscope in order to improve the accuracy of the colonoscopy.

FIG. 2 shows a photograph of a cap of the prior art with flanges (“endoring”), which is positioned behind the camera of the endoscope in order to improve the accuracy of the colonoscopy.

FIGS. 3 and 4 are schematic perspective views of a cap according to the present invention.

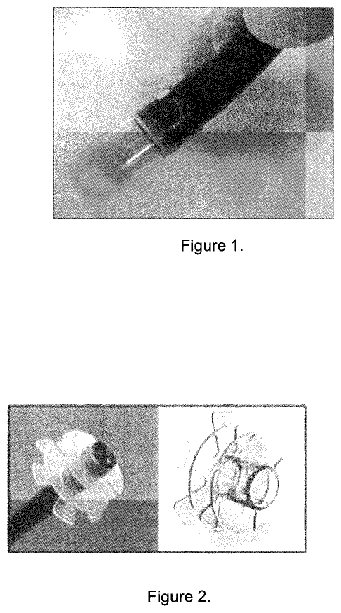

### DESCRIPTION

The present invention concerns a new endoscopy cap capable of improving the viewing of the hollow organs, owing to its capacity to distend effectively the folds of these hollow organs, allowing an expansion of the vision which the operator has of areas which are difficult to access, even when not directly facing the camera mounted on the endoscope.

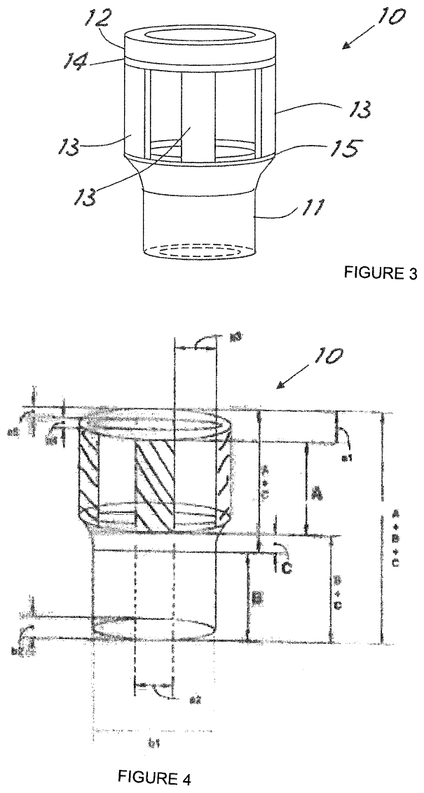

With reference to FIG. 3, the cap according to the present invention (indicated generally by 10) comprises:
a part or ring 11 for anchoring to the endoscopic probe (not shown), consisting of a ring of preferably plastic or silicone material, to be inserted mechanically onto the free end of the endoscopic probe;
an internally hollow ring 12, called “targeting ring”, made of preferably plastic material and intended to rest against the mucosa of the various parts of the gastro-intestinal tract and cause the distension of its surface and its folds, while maintaining within the said ring a homogeneous planar vision of the area located by the said targeting ring; and
a series of ties or arms 13 connecting together the anchoring ring 11 and the targeting ring 12, in turn produced from the same material, preferably plastic or silicone (or other type of material), having the function of keeping the two rings spaced at an ideal distance for maintaining the planar vision of the area enclosed by the targeting ring and also of the laterally visible area without image distortion due to the presence of plastic material or the like.

According to an embodiment of the invention, the cap device is characterized by: a structure open on the sides of said cap, with a number of ties or arms which fasten the part in contact with the mucosal surface to the collar for anchoring to the head of the endoscope; the presence of a transparent material which allows a perspective view of the entire visual field of the camera lens; the presence of a surface beyond the camera lens which produces the distension of the superficial mucosal structures, so as to allow total and precise viewing thereof.

Among the advantages of the cap device according to the invention the following may be mentioned:
the possibility of having almost complete lateral vision, interrupted only by the thickness of the ties or arms which support the cap in the longitudinal direction;
being able to maintain the clarity, quality and high definition of the endoscopy image also in the more lateral zones;
the reduction of the total surface area and gramme weight of the device; and
being able to dispense with the need for distension flanges for the cap devices.

According to the present invention, said anchoring ring 11 may have a diameter which varies preferably from 9 mm to 15 mm, more preferably from 12.6 mm to 13 mm.

According to the present invention, said targeting ring 12 may have a diameter which varies preferably from 12.5 mm to 17.5 mm, more preferably between 16.8 mm and 17.2 mm. According to the present invention, said ties or connecting arms 13 are preferably from 3 to 6 in number for each cap device, more preferably at least 4 ties or connecting arms for each cap device. According to an embodiment, said ties or connecting arms have a length preferably of between 2 mm and 10 mm, more preferably between 8 and 9 mm, and even more preferably a length of about 8 mm and a width of between about 4 and 6 mm, more preferably of about 4.2 mm.

The material from which the cap device according to the present invention is made is preferably a plastic or silicone material, which is biocompatible with the mucosa of the gastro-intestinal tract, with a good transparency, and which does not cause irritation upon coming into contact with the mucosa. Preferably, said plastic or silicone material is chosen from polymethyl methacrylate, composed of synthetic material belonging to the polymer family of (disposable) methacrylates or polycarbonate (preferably LEXAN 4404 which is more transparent and autoclavable at temperatures of up to 134° C.), also belonging to the polymer family.

Owing to the cap device according to the invention it is possible to obtain the distension of the intestinal folds and the mucous membranes of the hollow organs of the gastro-intestinal tract, improving significantly viewing during analysis and endoscopic analysis per se.

The cap device according to the invention may also be mounted on different types of endoscopic probes such as, for example, colonoscopes with diametral dimensions of between 9 mm and 15 mm or on gastroscopes with a thickness of between 9 mm and 15 mm. According to a preferred embodiment of the invention, the cap device is mounted on colonoscopes. In a typical endoscopic procedure a cap made according to the prior art is mounted on the free part of the endoscope before introducing the probe into the hollow organ of the patient. Following the introduction of the probe, when carrying out the endoscopic observation procedures during insertion or, rather, retraction or removal of the probe, the cap has the function of facilitating distension of the intestinal folds, allowing the endoscopy operator to assess the possible presence of polyps or lesions of the mucosa adjacent thereto. The presence of lateral surfaces of the cap, however, prevents most of the visual field being captured within the circle defined by the opening of the said cap, with the result that the endoscope operator must operate the endoscope with rotary-flexural movements in order to compensate for this deficiency, using the visual field restricted by the open part on the tip of the cap. The sides surfaces of the cap therefore act as real optical barriers, with the result that the endoscope operator must prolong examination, as though using an endoscope with a significantly reduced visual field.

By fitting a cap designed according to the present invention, instead, the additional rotary-flexural movements of the endoscope in order to compensate for reduction of the visual field are no longer necessary since the open side surface of the device does not form an obstacle or barrier preventing viewing of the mucosal surface, while retaining the properties of a mucosa distension device due to the presence of the distal ring and the arms of the said cap. The endoscopic procedure in this case is facilitated and accelerated considerably owing to the absence of obstacles to vision represented by the image deformation due to the infiltration of material which is not perfectly transparent (i.e. plastic material, with its characteristic vision deformation properties due to the type of material and its thickness), while leaving unchanged the capacity for distension of the intestinal mucosa and its folds.

In the preferred embodiment of the cap device according to the invention shown in the figure, a number of dimensional references are schematically shown, as described more fully below.

A: height of the targeting part which comprises the targeting ring 12 and ties or arms 13 and which may be made of plastic material (belonging to the polymer family of methacrylates), and where:
a1: diameter of the targeting ring 12;
a2: width of the ties or connecting arms 13;
a3: lateral openings for complete viewing of the mucosa between the arms 13;
a4: diameter of the hollow circular area inside the targeting ring 12 for any handle openings;
a5: thickness of the targeting ring.

A connecting ring 14 which joins the ties or arms 13 to the targeting ring 12 may also be present between the arms 13 and the targeting ring 12.

Still with reference to FIG. 4 the following are shown:

B: height of the ring or collar 11 for anchoring to the endoscope, composed of biocompatible silicone material, and where
b2: diameter of the ring 11 for anchoring to the endoscope;
b1: thickness of the anchoring collar or ring 11.

A further connecting ring 15 may also be present between the anchoring ring or collar 11 and the arms 13.

The connecting rings 14 and 15 may also be formed as one piece with the arms 13 so as to form an element for easier fixing of the anchoring rings, for example made of silicone material, and targeting ring 12 and/or arms, also made for example of highly transparent biocompatible plastic material.

As is clear from FIGS. 3 and 4, the anchoring ring may also have a form with variable diameter (in particular the conical top end with height C in FIG. 4) so as to adapt the diameter which is required for the targeting part to the diameter which is required for the anchoring collar or ring 11 in the endoscope fixing zone. This variable-diameter element may also form part of the connecting ring 15, instead of the anchoring ring 11.

At this point it is clear how the objects of the invention have been achieved, by providing an endoscope cap which allows suitable distension of the walls and satisfactory viewing from both the side and the front.

Obviously, the above description of an embodiment applying the innovative principles of the present invention is provided by way of example of these innovative principles and must therefore not be regarded as limiting the scope of the rights claimed herein. For example, the various parts of the cap may be made of materials different from those mentioned and also be combined with each other by means of various known systems, such as co-molding or gluing together, or may be made as a single piece of suitable material. The radial form and the proportions of the cap may also vary depending on the practical requirements, and radially projecting flanges may also be provided.

1. A cap device for endoscopic applications comprising:
an anchoring ring for attachment to an endoscopic probe, to be inserted mechanically on a free end of the probe,
a targeting ring, comprising an internally hollow ring for resting against a mucosa of various areas of a gastro-intestinal tract and causing distension of a surface thereof and its folds, maintaining inside the targeting ring a homogeneous planar vision of an area located by said targeting ring;
a series of connecting arms connecting the anchoring ring and the targeting ring, for keeping the anchoring ring and targeting ring spaced at a defined distance for maintaining the planar vision of the area located by the targeting ring and also of a laterally visible area without image distortion.

2. The cap device according to claim 1, characterized in that said anchoring ring has a diameter of between 9 mm and 15 mm.

3. The cap device according to claim 1, characterized in that said targeting ring has a diameter of between 12.6 mm and 17.5 mm.

4. The cap device according to claim 1, characterized in that said connecting arms have a length of between 7.5 mm and 10 mm.

5. The cap device according to claim 1, characterized in that said series of ties or connecting arms has a width of between 4 mm and 6 mm.

6. (canceled)

7. The cap device according to claim 1, where said plastic material is a plastic material biocompatible with the mucosa of the gastro-intestinal tract.

8. The cap device according to claim 1, wherein between said connecting arms and the targeting ring and/or between said connecting arms and the anchoring ring there is a connecting ring, optionally made as one piece with the connecting arms.

9. The cap device according to claim 1 wherein one or more of the anchoring ring, the targeting ring and/or the connecting arms comprise a plastic material, a silicone material and combinations thereof.

10. The cap device according to claim 1, wherein said anchoring ring has a diameter of between 12.5 mm and 13 mm.

11. The cap device according to claim 1, wherein said targeting ring has a diameter of between 16.8 mm and 17.2 mm.

12. The cap device according to claim 1, wherein said connecting arms have a length of between 8 mm and 9 mm.

13. The cap device according to claim 1, wherein said connecting arms have a length of about 8 mm.

14. The cap device according to claim 1, said connecting arms have a width of about 4.2 mm.

15. The cap device according to claim 9 where said plastic material is a plastic material biocompatible with the mucosa of the gastro-intestinal tract.

16. The cap device according to claim 15 where said plastic material is taken from the group consisting of polymethyl methacrylate, polycarbonate or mixtures thereof.
